(19) United States
(12) Patent Application Publication
(10) Pub. No.: US 2019/0321528 A1
(43) Pub. Date: Oct. 24, 2019

(54) PROSTHETIC PUMP AND DELIVERY SYSTEM

(71) Applicant: W. L. Gore & Associates, Inc., Newark, DE (US)

(72) Inventor: Kevin K. Coffman, Flagstaff, AZ (US)

(21) Appl. No.: 16/391,902

(22) Filed: Apr. 23, 2019

Related U.S. Application Data
(60) Provisional application No. 62/661,611, filed on Apr. 23, 2018.

(51) Int. Cl. A61M 1/10; A61M 1/12; A61F 2/24

(52) U.S. Cl. CPC A61M 1/1008; A61M 1/125; A61M 2205/3569; A61M 1/1086; A61F 2/2445; A61M 1/122

(57) ABSTRACT

Various aspects of the present disclosure are directed toward apparatuses, methods and systems for improving or assisting cardiac function of a patient. The apparatuses, methods and systems may include a support structure configured to extend across leaflets of an aortic valve of the patient. In addition, the support structure may be configured to removably couple a pump.

### CROSS-REFERENCE TO RELATED APPLICATION

This application claims the benefit of Provisional Application No. 62/661,611, filed Apr. 23, 2018, which is incorporated herein by reference in its entirety for all purposes.

### FIELD

The present disclosure relates generally to medical devices and more specifically to implantable cardiac assist devices and supporting structures configured to operate within a patient's vasculature and that can be minimally invasively delivered via a catheter.

### BACKGROUND

Cardiac assist devices (such as ventricular assist devices (VAD)) generally relate to systems that include a pump that assists heart function without replacing the heart in order to improve hemodynamics. Depending on the needs and demands of the patient, the pump may be placed outside the patient's body (extra- or para-corporeal devices), or within the patient's abdomen such as in the pericardial space beneath or above the diaphragm (intracorporeal device). Attempts have also been made to place such pumps within the patient's vasculature.

### SUMMARY

According to one example (“Example 1”) a medical device for improving or assisting cardiac function of a patient includes a support structure configured to extend across leaflets of a valve of the patient, the support structure having a delivery configuration and a deployed configuration; and one or more locating features arranged within an interior of the support structure and configured to removably couple a pump to the support structure in the deployed configuration.

According to another example (“Example 2”), further to the medical device of Example 1, the medical device also includes a pump is configured to drive blood flow through the support structure and supply blood flow to an aorta and couple to the one or more locating features of the support structure.

According to another example (“Example 3”), further to the medical device of Example 2, the support structure is configured to removably couple the pump after the support structure is deployed from the delivery configuration to the deployed configuration and the pump includes one or more engagement elements configured to lock within the one or more locating features of the support structure.

According to another example (“Example 4”), further to the medical device of Example 3, the pump and the support structure form a seal therebetween to stop blood flow between the pump housing and the support structure.

According to another example (“Example 5”), further to the medical device of Example 3, the support structure is configured to suspend the pump within the support structure to allow blood flow about the pump.

According to another example (“Example 6”), further to the medical device of any one of Examples 2-5, the support structure is configured to pin the leaflets to heart tissue in an open position to minimize interference with the pump.

According to another example (“Example 7”), further to the medical device of any one of Examples 2-6, the medical device also includes a controller configured to power the pump and a drive line coupled to the pump and the controller and configured to deliver power to the pump.

According to another example (“Example 8”), further to the medical device of Example 7, the drive line is configured to route through one of the left or right subclavian arteries.

According to another example (“Example 9”), further to the medical device of any one of Examples 1-8, the support structure includes at least one of a stent and a graft configured to collapse to the delivery configuration and engage and interface with the leaflets of the valve upon expansion of the support structure to the deployed configuration.

According to one example (“Example 10”), a modular system for assisting cardiac function of a patient includes a support structure configured to deploy at a target treatment region within the patient, the support structure including one or more locating features arranged within an interior of the support structure; a pump including one or more engagement elements configured to removably couple to the locating features of the support structure after deployment of the support structure at the target treatment region; and a power source configured to power the pump to drive blood flow through the support structure.

According to another example (“Example 11”), further to the system of Example 10, the power source includes a controller configured to power the pump and a drive line removably coupled to the pump and the controller configured to deliver the power to the pump.

According to another example (“Example 12”), further to the system of Example 11, the drive line is configured to route through one of the left or right subclavian arteries and couple to the pump after deployment of the pump within the support structure.

According to another example (“Example 13”), further to the system of any one of Examples 10-12, the power source includes an extracorporeal control system configured to control operation of the pump and to wirelessly power the pump.

According to another example (“Example 14”), further to the system of Example 13, the extracorporeal control system includes a transcutaneous energy transmission system configured to wireless transmit energy to the pump.

According to another example (“Example 15”), further to the system of Example 14, the pump includes an antenna configured to receive the transcutaneous energy transfer.

According to one example (“Example 16”), method of improving or assisting cardiac function of a patient includes deploying a pump system across leaflets of a valve of the patient, the pump system including a support structure having one or more locating features arranged within an interior of the support structure and a pump including one more engagement features configured to removably couple to the one or more locating features of the support structure; and operating the pump to drive blood flow through the support structure.

According to another example (“Example 17”), further to the method of Example 16, the operating the pump includes driving blood across the pump and into an aorta.

According to another example (“Example 18”), further to the method of Example 17, operating the pump includes drawing blood from a left ventricle and across the pump into the aorta.

According to one example (“Example 19”), a method of delivering a medical device for assisting cardiac function of a patient to a target location includes deploying a support structure a target treatment region within the patient, the support structure including one or more locating features arranged within an interior of the support structure; arranging a pump within the support structure, the pump including one or more engagement elements; and coupling the pump to the support structure by engaging the locating features of the support structure and the engagement elements of the pump.

According to another example (“Example 20”), further to the method of Example 19, the method also includes coupling a power source to the pump.

According to another example (“Example 21”), further to the method of Example 20, the power source includes a controller configured to power the pump, and coupling the power source to the pump includes coupling a drive line to the pump and the controller configured to deliver the power to the pump.

According to another example (“Example 22”), further to the method of Example 21, coupling the drive line to the pump includes routing the driveline through one of the left or right subclavian arteries and couple to the pump after coupling the pump to the support structure.

According to another example (“Example 23”), further to the method of Example 20, the coupling a power source to the pump includes wirelessly coupling an extracorporeal control system to the pump to control operation of the pump and to wirelessly power the pump

According to another example (“Example 24”), further to the method of Example 19, deploying the pump system includes arranging the support system through one of a femoral access, a subclavian access, or transcaval access.

While multiple embodiments are disclosed, still other embodiments will become apparent to those skilled in the art from the following detailed description, which shows and describes illustrative examples. Accordingly, the drawings and detailed description are to be regarded as illustrative in nature and not restrictive.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of a system including a support structure and a pump, according to some embodiments;

FIG. 2 is an illustration of a support structure according to some embodiments;

FIG. 3 is an illustration of a pump, according to some embodiments;

FIG. 4A is a cross sectional view of the support structure shown in FIG. 2, taken along line 4A-4A;

FIG. 4B is a cross sectional view of the pump shown in FIG. 3, taken along line 4B-4B;

FIG. 5 is a cross sectional view of a human heart with the system of FIG. 1 positioned therein in a collapsed, delivery state, according to some embodiments;

FIG. 6 is a cross sectional view of a human heart with the system of FIG. 1 positioned therein in an expanded, deployed state, according to some embodiments;

FIG. 7A is an illustration of a support structure in a denested configuration, according to some embodiments;

FIG. 7B is an illustration of the support structure of FIG. 7A in a nested configuration, according to some embodiments;

FIG. 8 is a cross sectional view of the support structure shown in FIG. 7B taken along line 8-8;

FIG. 9 is an illustration of various additional configurations for the support structure, the pump, and the retention element, according to some embodiments; and

FIG. 10 is a side view an example system, according to some embodiments.

### DETAILED DESCRIPTION

### Definitions and Terminology

As the terms are used herein with respect to ranges of measurements “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

This disclosure is not meant to be read in a restrictive manner. For example, the terminology used in the application should be read broadly in the context of the meaning those in the field would attribute such terminology.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

A coordinate system is presented in the Figures and referenced in the description in which the “Y” axis corresponds to a vertical direction, the “X” axis corresponds to a horizontal or lateral direction, and the “Z” axis corresponds to the interior/exterior direction.

The term “leaflet” as used in the context of prosthetic valves is generally a flexible component operable to move between an open and closed position under the influence of pressure differentials. For example, in operation, the leaflets open when an inflow fluid pressure exceeds an outflow fluid pressure and close when the outflow fluid pressure exceeds the inflow fluid pressure. In a closed position, the leaflet, alone or in combination with one or more other leaflets, operates to substantially restrict or obstruct (or alternatively completely obstruct) retrograde flow through the prosthetic valve. Thus, it will be appreciated that, in some instances, coaptation of adjacent leaflets may operate to completely block the flow of fluid (e.g., blood) through the prosthetic valve, while in other instances coaptation of adjacent leaflets may operate to block less than all of the flow of fluid (e.g., blood) through the prosthetic valve. In some embodiments, the leaflets include a free edge, and the free edges of adjacently situated leaflets coapt under the influence of outflow fluid pressure, thereby closing the valve so as to restrict or obstruct fluid from flowing retrograde through the prosthetic valve.

### Description of Various Embodiments

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward systems and methods for use in association with the cardiac function of the heart. The systems generally include a pump and a support structure (e.g., stent, stent graft, graft, fluid flow conduit) configured to support and maintain a position of the pump during its operation within the patient's vasculature. Disclosed systems and methods also include a delivery system configured for transcatheter delivery of the pump and the support structure.

The present disclosure relates to systems and methods for improving or assisting the cardiac function of the heart. The disclosed systems and methods generally include a pump and a supporting structure including a stent that is configured to support and maintain a position of the pump during its operation within the patient's vasculature. The system is configured to be implanted within the patient's vasculature such that one or more of the pump, the pump housing, and the support structure extend across the native valve. The disclosed systems and methods also include a delivery system configured for transcatheter delivery of the pump and the support structure.

In the instant disclosure, the examples are primarily described in association with transcatheter cardiac applications involving the aorta and the aortic valve (also referred to herein as left ventricular assist), although it should be readily appreciated that the various embodiments and examples discussed herein can be applied in association with any known uses of ventricular assist devices, including for use within other regions of the heart, such as for use in association with the pulmonary valve (e.g., in association with a right ventricular assist application).

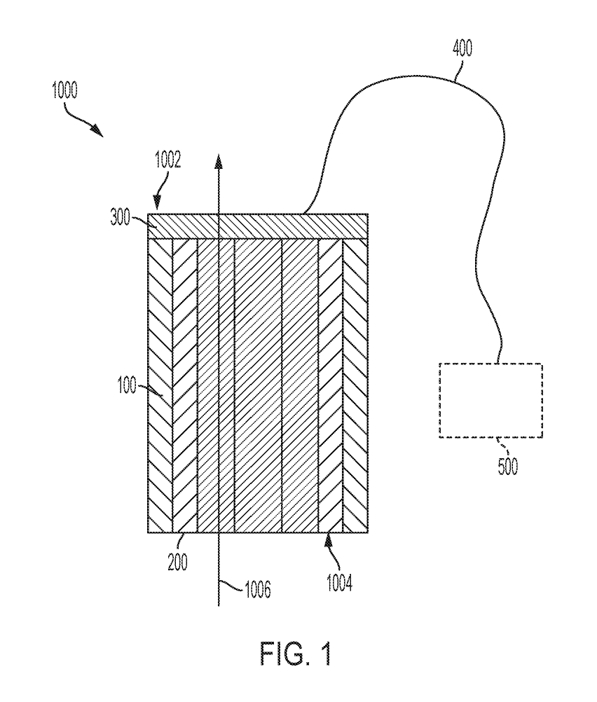

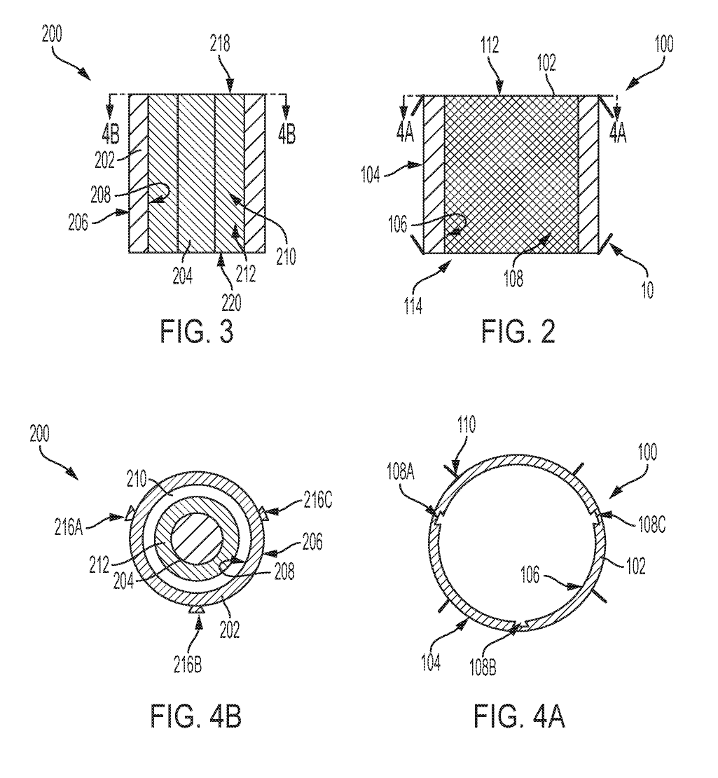

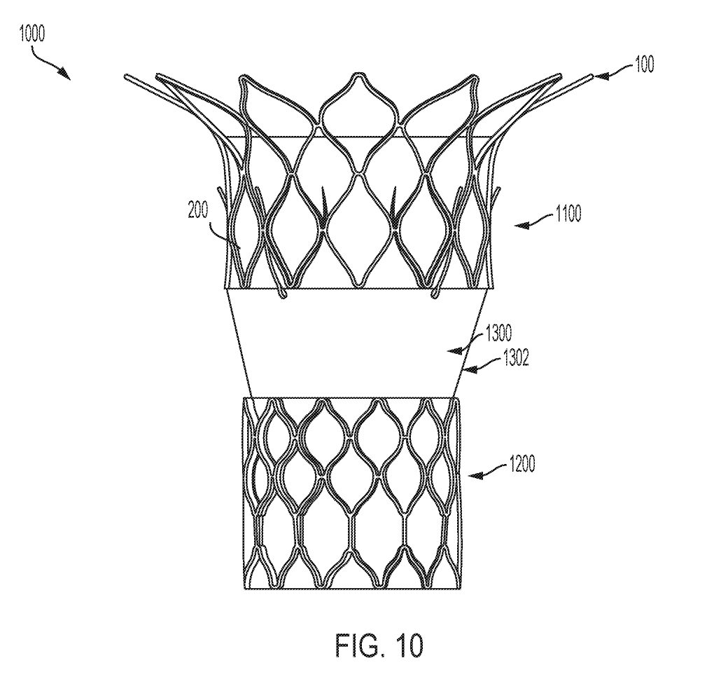

As shown in FIG. 1, a system 1000 according to various embodiments includes a support structure 100, a pump 200 disposed at least partially within the support structure 100, and a retention element 300 configured to help maintain a position of the pump 200 within the support structure 100. The retention element 300 is an optional element that may engage and interface with the support structure 100 to maintain the coupling between the pump 200 and the support structure 100 as described in further detail below. With reference now to FIG. 2, the support structure 100 generally includes a stent body 102 defining an exterior 104 and an interior 106. The stent body 102 may be generally cylindrically shaped and configured to adopt a profile consistent with the vasculature within which is it deployed and expanded. In some examples, the stent body 102 is defined by a plurality of interconnected strut elements 108, as those of skill in the art will appreciate. Examples of suitable stent bodies similar to those described above are discussed in further detail below with reference to FIG. 10 and further discussion of which can be found in application Ser. No. 16/129,779 (claiming priority to Provisional Application Ser. No. 62/579,762), entitled “TELESCOPING PROSTHETIC VALVE AND DELIVERY SYSTEM,” filed by Applicant.

The support structure 100 may comprise, such as, but not limited to, elastically deformable metallic or polymeric biocompatible materials. The support structure 100 may comprise a shape-memory material, such as nitinol, a nickel-titanium alloy. Other materials suitable for the support structure 100 include, but are not limited to, other titanium alloys, stainless steel, cobalt-nickel alloy, polypropylene, acetyl homopolymer, acetyl copolymer, other alloys or polymers, or any other biocompatible material having adequate physical and mechanical properties to function as the support structure 100, as described herein. The support structure 100 may therefore be self-expanding and/or may be balloon expandable. That is, in various examples, the support structure 100 may be transitionable between a collapsed delivery configuration and an expanded deployed configuration.

Additionally, in some embodiments, the support structure 100 may further include a graft material disposed thereabout (e.g., such as about an interior of or an exterior of the support structure 100). In various embodiments, graft materials in stent-grafts can include, for example, expanded polytetrafluoroethylene (ePTFE), polyester, polyurethane, fluoropolymers, such as perfluoroelastomers and the like, polytetrafluoroethylene, silicones, urethanes, ultra high molecular weight polyethylene, aramid fibers, and combinations thereof. Other embodiments for a graft member material can include high strength polymer fibers such as ultra-high molecular weight polyethylene fibers (e.g., Spectra®, Dyneema Purity®, etc.) or other fibers (e.g., Technora®, etc.). Some embodiments may comprise of a graft material only partially disposed about the support structure frame. The graft member can include a bioactive agent. In one embodiment, an ePTFE graft includes a carbon component along a blood contacting surface thereof. Any graft member that can be delivered in a patient to a treatment site is in accordance with the present disclosure.

The exterior 104 of the support structure 100 is generally configured to engage and interface with a patient's anatomy to maintain a position of the support structure 100 within the patient's anatomy, as those of skill will appreciate. For instance, in some examples, the stent body 102 includes one or more anchoring elements 110 that are configured to extend from the exterior 104 of the stent body 102 such that the anchoring elements 110 are operable to engage tissue. The interior 106 of the support structure 100, on the other hand, is configured to engage and interface with the pump 200. For example, as discussed in greater detail below, the system 1000 may be configured such that the pump 200 can be removably coupled with the support structure 100. Removably coupling the pump 200 with the support structure 100 allows for a modular system in that the pump 200 may be coupled with the support structure 100 after the support structure 100 has been delivered and deployed within the patient's vasculature and/or that the pump 200 may be removed from the patient's vasculature without also requiring removal of the support structure 100 (e.g., such that the pump 200 may be replaced and/or such that removal of the system 1000 may be done minimally invasively).

Turning back to FIG. 1, the pump 200 is generally configured to drive or otherwise cause blood to flow across the pump 200 from an inflow side 1004 of the system 1000 to an outflow side 1002 of the system, such as along a direction of arrow 1006. The pump mechanism (also referred to herein as a pump drive) of the pump 200 may operate in accordance with known principles, including centrifugal-action pumps, as well as others. For instance, in some examples the pump 200 may include a worm-style drive mechanism, impeller, or any other suitable drive configuration known in the art. In other examples, the pump 200 may include a pneumatic bladder, driven for example by an oscillating signal, including a capability of providing circulatory support synchronously with the native cardiac cycle, as those of skill in the art will appreciate. The pump housing is configured to interface and engage with the support structure 100, as explained further below.

For example, as shown in FIG. 3, the pump 200 may generally include a pump housing 202 and a pump drive element 204. The pump housing 202 generally defines an exterior 206 and an interior 208. The exterior 206 of the pump housing 202 is configured to engage and interface with the interior 106 of the support structure 100 such that the pump 200 can be coupled with the support structure 100. The interior 208 of the pump housing 202 is configured to house or accommodate the pump drive element 204 such that the pump drive element 204 can move relative to the pump housing 202 to cause blood to flow through the pump 200. In some examples, blood travels through the pump 200 within an annular space 210 that is defined between the pump drive element 204 and the pump housing 202, although other pump configurations are contemplated and fall within the scope of the present disclosure provided that the pump housing can be configured to interface and engage with the support structure 100. Thus, although the pump drive element 204 shown in FIG. 3 includes a worm drive having a helical flange extending about a central shaft (e.g., an impeller configuration), the application should not be understood to be limited to such configuration, but should instead be understood to be operable with other pump drive configurations. In certain instances, the pump housing 202 may be one or more retention elements 300 that facilitate positioning of the pump 200 within the support structure 100.

As shown in FIG. 1 and as mentioned above, the system 1000 may include one or more retention elements 300 (also referred to herein as a cap) that is configured to help maintain a position of the pump 200 within the support structure 100. The retention element 300 is thus configured to engage and interface with the support structure 100 to maintain the coupling between the pump 200 and the support structure 100. The retention element 300 may couple to the stent support element 100 by way of one or more clips, tethers, channels, threads, or other suitable mechanical means. In other instances, as described in further below relative to FIGS. 2-4, the system 1000 may include engagement elements and corresponding pump locating features to couple the pump 200 to the support structure 100. The engagement elements and corresponding pump locating features may be in addition to or in place of the retention element 300.

In some examples, the retention element 300 may be coupleable to the support structure 100 after the support structure 100 has been delivered and deployed within the patient's vasculature. Similarly, in some examples, the retention element 300 may be removed from the system 1000 without also requiring removal of one or more of the support structure 100 and the pump housing 202 of the pump 200. Thus, in some examples, the retention element 300 may be removed from the patient's anatomy while one or more of the support structure 100 and the pump 200 remain deployed within the patient's vasculature.

In some embodiments, the system 1000 further includes a driveline 400. The driveline 400 is a cable assembly that operates to electrically couple a controller 500 located external to the patient's anatomy with the manual pump 200, as those of skill will appreciate. As discussed, the driveline 400 may be routed through the patient's vasculature and then out through the skin to where it is coupled with the controller 500. The controller 500 is a module that is configured to control the operation of the pump 200, as those of skill in the art will appreciate, and thus may operate according to known methods.

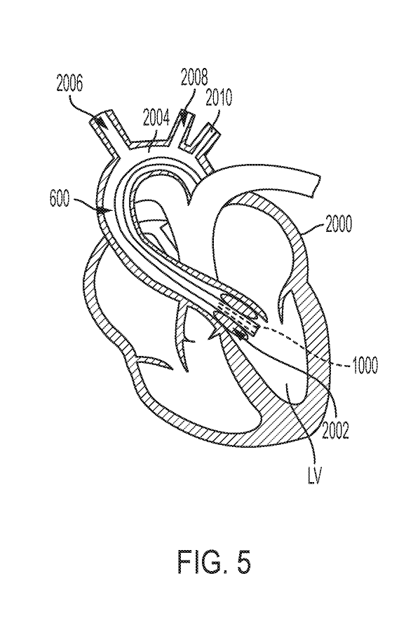

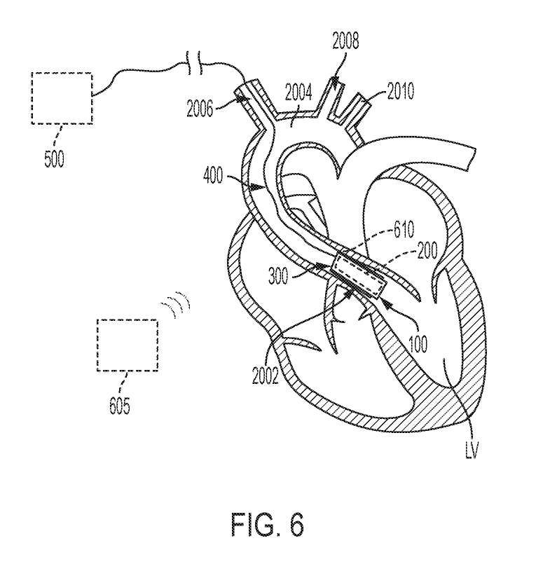

In some examples, the driveline 400 may be routed through one of the left or right subclavian arteries 2010 and 2006 (FIGS. 5 and 6), or the left common carotid artery 2008 (FIGS. 5 and 6) to a subclavian or other associated access. Alternatively, the driveline 400 may be routed through the descending aorta to a femoral or other associated access. In some examples, the driveline 400 is routed through the retention element 300. In some examples, the driveline 400 is integral to the retention element 300 and includes one or more connectors such that when the retention element 300 is coupled to the support structure 100, the driveline 400 is electrically coupled with the pump 200.

As mentioned above, in various embodiments, the pump 200 is receivable within the support structure 100. As shown in FIGS. 4A and 4B, each of the pump 200 and the support structure 100 include complementary features that facilitate the coupling of the support structure 100 with the pump 200. FIG. 4A is a cross sectional view of the support structure 100 taken along line 4A-4A of FIG. 2. FIG. 4B is a cross sectional view of the pump 200 taken along line 4B-4B of FIG. 3. As shown in FIG. 4A, the support structure 100 includes a plurality of pump locating features 108a, 108b, and 108c. In this illustrated example, the pump locating features 108a-108c are channels or recesses that extend longitudinally along a longitudinal axis of the support structure 100. In some examples, the pump locating features 108a-108c extend parallel to the longitudinal axis of the support structure 100. In some examples, one or more of the pump locating features 108a-108c extend along less than all of the length of the support structure 100. That is, in some examples, the pump locating features 108a-108c extend only partially between the first end 112 and the second end 114 of the support structure 100. In some such examples, one or more of the pump locating features 108a-108c terminates at a location between the first and second ends 112 and 114. This termination of the one or more channels or recesses of the pump locating features 108a-108c operates as an abutment against which the pump housing 202 of the pump 200 can sit.

As explained further below, such a configuration provides that the pump housing 202 of the pump 200 may only be inserted into and removed from the support structure 100 in a unidirectional manner. For instance, when inserted into the support structure 100, the pump 200 can be advance longitudinally along the support structure 100 until the pump housing 202 engages the termination point of the one or more channels or recesses of the pump locating features 108a-108c. Moreover, when being removed from the support structure 100, the pump 200 can only be withdrawn in a direction opposite from that direction in which the pump 200 was advanced when it was coupled to the support structure 100. Securing the pump 200 within the support structure 100 in such a manner operates to prevent the pump 200 from being drawn through the support structure 100 and into the left ventricle (LV), for example. On the other hand, the engagement between the support structure 100 and the surrounding tissue (e.g., the heart/vessel wall tissue) operates to prevent the support structure 100 from being drawn into the left ventricle.

As mentioned above, the pump housing 202 generally includes one or more features that are complementary of the pump locating features 108a-108c of the support structure 100. With reference now to FIG. 4B, the pump housing 202 is shown as including a plurality of engagement elements 216a, 216b, and 216c. As shown, the engagement elements 216a-216c are features that protrude from the exterior of the pump housing 202. The engagement elements 216a-216c extend longitudinally along the exterior 206 of the pump housing 202, such as parallel to a longitudinal axis of the pump housing 202. In some examples, the engagement elements 216a-216c extend between the first end 218 and the second end 220 of the pump housing 202. In some examples, one or more of the engagement elements 216a-216c may extend beyond (or alternatively short of) one or more of the first and second ends 218 and 220 of the pump housing 202. The engagement elements 216a-216c are generally complementary in shape, size, and location and orientation of the pump locating features 108a-108c such that the engagement elements 216a-216c can be received within the pump locating features 108a-108c.

As sown in FIGS. 4A and 4B, the engagement elements 216a-216c are formed as positive dovetail features while the pump locating features 108a-108c are formed as the complementary negative dovetail features. Additionally, the engagement elements 216a-216c are shown as being evenly distributed circumferentially about the exterior 206 of the pump housing 202, while the pump locating features 108a-108c are similarly evenly distributed circumferentially about the interior 106 of the support structure 100.

It is to be appreciated that the interaction between the engagement elements 216a-216c and the pump locating features 108a-108c operates to help locate the pump 200 within the support structure 100. For instance, the engagement between engagement elements 216a-216c and the pump locating features 108a-108c (the combination of which are referred to herein as alignment features) helps to align the pump 200 longitudinally with respect to the support structure 100. Likewise, the engagement between engagement elements 216a-216c and the pump locating features 108a-108c helps to align the pump 200 coaxially with the support structure 100.

Additionally, in various examples, this interaction also operates to prevent pitch/yaw/roll (e.g., rotation relative to the longitudinal axis of the support structure 100) of the pump housing 202 relative to the support structure 100 during operation of the system 1000, which provides the constraint necessary to allow the pump 200 to operate to drive blood flow across the pump 200 (e.g., the pump drive 204 can rotate or be rotated relative to the pump housing 202 without the pump housing 202 also rotating).

In various examples, with the pump 200 properly aligned and seated within the support structure 100, the pump housing 202 and the support structure 100 form a seal therebetween such that blood cannot flow between the pump housing 202 and the support structure 100. In some examples, the pump housing 202 is suspended within the support structure 100 such that blood can flow either through/across the pump drive element 204, or around the pump housing 202. Such a configuration allows for blood flow around the pump in the case of a pump failure, and additionally provides favorable hemodynamics with regard to hemolysis and perfusion of the coronary arteries. In some examples, bypass blood flow (e.g., blood flow around the pump 200 may be facilitated by the support structure 100, itself. For instance, in some examples, the support structure 100 may include an open celled stent structure, wherein the pump 200 is positioned within or suspended by the open celled stent support structure, which allows for blood to flow through and around the pump 200 (e.g., through the open cells of the stent support structure).

It is also to be appreciated that while the support structure 100 and the pump 200 shown in FIGS. 4A and 4B include complementary alignment features that are in the shape of dovetails, various other sizes and shapes of such features are envisioned and can be implemented without departing from the spirit or scope of the present disclosure. For example, the dovetail geometry may be replaced with one or more of various alternative geometries, including but not limited to, triangles, squares, loops, and polygons. Similarly, though the FIGS. 4A and 4B show three evenly distributed (e.g., positioned 120 degrees away from each other) alignment features (e.g., engagement elements 216a-216c and pump locating features 108a-108c), as little as one or two such alignment features may be used, or more than three such alignment features may be used. Likewise, where more than one alignment feature is used, such alignment features need not be evenly distributed about the interior/exterior of the support structure 100 and the pump housing 202 and need not be of the same size and shape.

It should also be appreciated that while the alignment features shown in FIGS. 4A and 4B extend longitudinally along the support structure 100 and the pump housing 200, the alignment features may alternatively be arranged in a helical pattern or other keyed pattern. In such an alternative configuration, the pump 200 is coupleable with the support structure 100 by aligning the helical or keyed alignment features of the pump 200 and the support structure 100 with one another and then rotating the pump 200 and the support structure 100 relative to one another, such as about the longitudinal axis of the support structure 100, for example.

In certain instances, the support structure 100 may be delivered to a target location prior to the pump 200. After the support structure 100 is arranged at the target location (e.g., as shown in FIGS. 5-6), the pump 200 may be separately advanced and seated within the support structure 100. The pump 200 may be advanced within the support structure 100 by circumferentially aligning the engagement elements 216a-216c and the pump locating features 108a-108c, and longitudinally sliding the pump 200 within the support structure 100. The pump 200 may be locked or keyed within the support structure 100 by the natural forces of the pump 200. Torque from the operation of the pump 200, for example, may lock the pump 200 within the support structure 100 through engagement of the engagement elements 216a-216c and the pump locating features 108a-108c.

It should also be appreciated that while the support structure 100 and the pump 200 shown in FIGS. 4A and 4B are shown with the alignment features protruding from the exterior 206 of the pump housing 202 and as channels or recesses along the interior 106 of the support structure 100, in some other examples, the alignment features may protrude from the interior 106 of the support structure 100 and be formed as recesses or channels or other features appropriate to receive such protruding alignment features along the exterior 206 of the pump housing 202. Alternatively, the support structure 100 and the pump housing 200 may each include a combination of alignment features that protrude therefrom and that are formed as recesses or channels therein.

Turning now to FIG. 5, the system 1000 is shown in a delivery configuration during a delivery procedure in which the system 1000 is delivered to the aortic valve 2002 within the patient's heart 2000. In other instances, the system 1000 may be arranged across another valve (e.g., mitral) of a patient. The system 1000 is shown in FIG. 5 disposed about the distal end of a catheter 600. In some examples, the system 1000 in the compacted, delivery state, or configuration, is able to be received inside of a constriction sheath (not shown), and then extended or expanded upon withdrawal or removal of the sheath. Examples of suitable delivery systems similar to those described above can be found in application Ser. No. 16/129,779 (claiming priority to Provisional Application Ser. No. 62/579,762), entitled “TELESCOPING PROSTHETIC VALVE AND DELIVERY SYSTEM,” filed by Applicant and referred to above, as well as in application Ser. No. 16/129,657 (claiming priority to Provisional Application No. 62/579,756), entitled “TRANSCATHETER DEPLOYMENT SYSTEMS AND ASSOCIATED METHODS,” filed by Applicant.

For example, the catheter 600 can include a sheath (not shown) with at least the support structure 100 mounted thereon. The pump 200 may be delivered separately using another catheter or the pump 200 may be arranged with the support structure 100 on the catheter 600. The support structure 100 (and pump 200 in the instances where the pump 20 is delivered within the support structure 100) is maintained in a collapsed configuration by the catheter 600. It should be noted that the sheath (not shown) or other features, such as constraining sleeves or jackets (not shown), can additionally or alternatively be employed along one or more portions of the support structure 100 to assist with maintaining the support structure 100 in a collapsed configuration.

In various examples, the system 1000 may be collapsible and deliverable as a preassembled unit. That is, in some examples, prior to collapsing the system 1000 onto the delivery catheter 600, the pump 200 may be coupled with the support structure 100, the driveline 400 may be electrically coupled to the pump 200, and the retention element 300 may be coupled with the support structure 100 to secure the pump 200 within the support structure 100. Thereafter, the system 1000 may be collapsed onto the delivery catheter 600 and maintained in a constricted delivery state.

Alternatively, in some examples, one or more components of the system 1000 may be assembled in situ. That is, in some examples, the system is configured such that one or more of the components of the system 1000 are coupled to one or more other components of the system 1000 after a portion of the system 1000 has been deployed within the patient's vasculature. For instance, in some examples, the system 1000 may be configured such that the support structure 100 is delivered to a target treatment region within the patient's anatomy (e.g., adjacent or across the aortic valve), and such that the pump 200 is subsequently coupled with the support structure 100 after the support structure 100 has been deployed (e.g., expanded within the vasculature). In some such examples, the support structure 100 and the pump 200 may be delivered on the same catheter in a decoupled state. In some other examples, the support structure 100 and the pump 200 may be delivered on different catheters, wherein the pump 200 is delivered subsequent to the delivery and deployment of the support structure 100. Similarly, it will be appreciated that the retention element 300 and/or the driveline 400 may be delivered with the same catheter as the support structure 100 and/or the pump 200, or may be delivered with one or more different catheters.

In various examples, with the system 1000 compacted or collapsed to the delivery state, the system 1000 is advanceable to a position within the aorta 2004 (also referred to herein as a landing position), wherein the system 1000 extends across the leaflets of the aortic valve 2002 (e.g., from an upstream side to a downstream side), as shown. This landing position within the aorta 2004 may be accessed through a femoral, a subclavian access, transcaval, or other suitable vascular access positions. When the system 1000 is positioned across the aortic valve 2004, the support structure 100 is situated such that it will engage and interface with the leaflets of the aortic valve upon deployment expansion of the support structure 100.

For example, FIG. 6 shows the system 1000 when the support structure 100 expanded against the leaflets of the aortic valve 2004. In particular, FIG. 6 shows the support structure 100 expanded such that the leaflets of the native aortic valve 2004 are pinned between the support structure 100 and the heart/vessel tissue. Pinning the native leaflets between the support structure 100 and the heart/vessel tissue operates to pin the native valve in an open position, which helps minimize a possibility that the native leaflets interfere with the operation of the pump 200. A properly designed pump 200 can be disposed across the aortic valve and operate to supply blood flow to both the aorta and to the coronary arteries. By comparison, a pump 200 placed distally to the aortic valve (e.g., fully within the aorta) and coronary ostia may encounter difficulties in promoting perfusion of the coronary arteries.

As mentioned above, it is to be appreciated that the support structure 100 may be self-expanded into the position shown in FIG. 6, and/or may be expanded into the position shown in FIG. 6 via an expandable balloon coupled to the catheter 600. Illustrations and examples of balloon expanding catheters and balloon expandable devices can be found in U.S. Pat. No. 4,776,337, entitled “EXPANDABLE INTRALUMINAL GRAFT, AND METHOD AND APPARATUS FOR IMPLANTING AN EXPANDABLE INTRALUMINAL GRAFT,” filed by on Jun. 26, 1986.

As shown in FIG. 6, the pump 200 is situated within the deployed support structure 100 such that the pump 200 is operable to pump or drive blood across the pump 200 and into the aorta and out into the vasculature of the body. That is, with the system 1000 in the deployed configuration, the pump 200 can be operated to draw blood from the left ventricle, blood across the pump 200, and into the aorta and out through the vasculature of the body.

As mentioned above, in various examples, the driveline 400 may be advanced through the vasculature (e.g., subclavian, femoral) and out through a percutaneous access site such that the driveline 400 may be coupled with a controller 500. As shown in FIG. 6, the driveline 400 extends distally from the retention element 300 toward and into the right subclavian artery 2006, such that the driveline 400 can be extended through a percutaneous access site (e.g., in the chest or shoulder) and coupled with a controller 500. In some examples, a second catheter or snare catheter may be utilized to snare or capture the driveline 400 and subsequently draw the driveline 400 through the desired portion of the vasculature. For example, a second catheter (not shown) may be routed through the right subclavian artery 2006 (e.g., such as through a percutaneous access site in the chest or in the shoulder) and subsequently routed into the aorta 2004 where the second catheter can be used to snare or capture the driveline 400 and draw the driveline 400 into the right subclavian artery 2006 and subsequently out through the percutaneous access site. Examples of snare catheters similar to those described above can also be found in U.S. application Ser. No. 15/591,755, entitled “FILTER AND OCCLUDER SYSTEMS AND ASSOCIATED METHODS AND DEVICES,” filed by Applicant hereof on May 10, 2017, and U.S. Pat. No. 8,992,545, entitled “IMPLANT-CATHETER ATTACHMENT MECHANISM USING SNARE AND METHOD OF USE,” filed by Applicant hereof on May 10, 2017, the entire contents of which are incorporated herein by reference.

For example, a snare device may include a snare wire and the distal end of the snare device forms a loop that catches the driveline 400. The snare device may be contained in a side lumen of the delivery system. The snare device may readily be released from the driveline 400 by advancing the snare wire until the loop unhooks from the driveline 400.

In some alternative embodiments, the system 1000 may be configured to operate without the need for the driveline 400, or the driveline 400 need not extend extracorporeally. That is, in some examples, an extracorporeal control system 605 may be configured to both control the operation of the pump, and to power the pump wirelessly (e.g., through a transcutaneous energy transmission system). In some examples, the extracorporeal control system 605 may be configured for transcutaneous energy transmission and may be accomplished through known means of transcutaneous energy transmission, such as those described in U.S. Pat. No. 6,400,991. Such a configuration eliminates distance of the driveline 400 route through the vasculature (e.g., if the energy transmission system is implanted subcutaneously) or eliminate the need for routing the driveline 400 out through a percutaneous access site, which can help minimize a risk for infection. In some examples, the driveline 400 may be configured to be unplugged or decoupled from the pump 200 at its junction with the pump 200. In some examples, decoupling the driveline 400 from the pump 200 includes decoupling or removing the retention element 300. In some examples, the system 1000 may include an antenna 610 that is configured for transcutaneous energy transfer (“TET”). In some examples, the antenna 610 may be incorporated into the retention element 300 or support structure 100 and may be selectively removable or replaceable. In some examples, the extracorporeal control system 605 may be an extracorporeal TET component maybe worn around the torso similar to a standard heart rate monitor, and additionally coupled to a power source (e.g., wall unit or high capacity battery) such that the extracorporeal TET component is operable to transmit energy transcutaneously to the antenna 610. In certain instances, the extracorporeal control system 605 may be subcutaneously implanted.

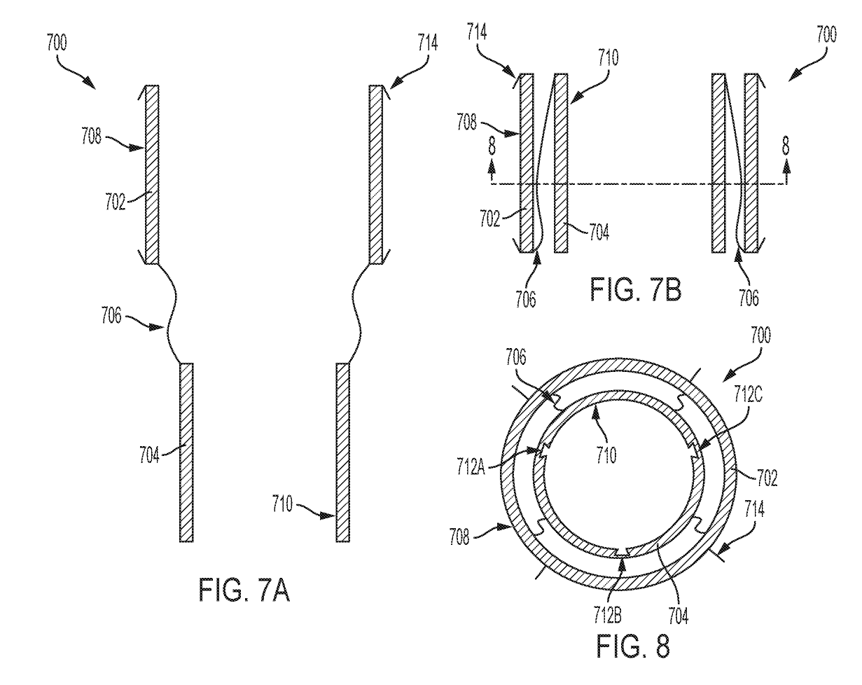

In some examples, the system 1000 may include a support structure having a plurality of sections including at a first section configured to engage and interface with the surrounding tissue and a second section that is nestable within the first section and that is configured to engage and interface with the pump 200. For example, turning now to FIGS. 7A, 7B, and 8, a support structure 700 is shown as including a first section 702 and a second section 704 that is nestable within the first section 702. The first and second sections may be coupled together via one or more linking elements 706. Moreover, one or more of the first and second sections 702 and 704 may include a plurality of strut elements (not shown for clarity purposes) consistent with the configuration of the strut support element 100 illustrated and described herein. FIG. 7A shows the support structure 700 in an unnested or denested configuration wherein the first and second sections 702 and 704 are unnested. FIG. 7B shows the support structure 700 in a nested configuration where the second section 704 is nested within the first section 702. In various examples, the second section 704 may be nested within the first section 702 by applying a force to the second section 704 to draw the second section 704 into the first section 702. FIG. 8 is a cross sectional view of the support structure shown in FIG. 7B taken along line 8-8.

As shown, in the nested configuration the first section 702 defines an exterior 708 of the support structure 700, while the second section 704 defines an interior 710 of the support structure 700. In various examples, the second section 704 is configured to accommodate, engage, and interface with the pump 200 in the nested configuration. In some examples, the second section 704 is also configured to accommodate, engage, and interface with the pump 200 in the denested configuration.

As shown in FIG. 8, the second section 704 includes a plurality of pump locating features 712a, 712b, and 712c consistent in form and function to the plurality of pump locating features 108a-108c, discussed above. Thus, it is to be appreciated that the second section 704 may be similar in form and function to the support structure 100 discussed above in terms of the alignment features between the pump 200 and the support structure 100. The second section 704 may be comprised of any of the materials suitable for the support structure 100, discussed above.

The first section 702, on the other hand, may be similar in form and function to the support structure 100 discussed above in terms of the anchorability of the support structure 100 within the heart/vessel wall tissue. For example, the first section 702 may be expandable (e.g., self-expanding and/or balloon expandable) and may thus be comprised of any of the materials suitable for the support structure 100, discussed above. Similarly, in various examples, the first section 702 may include one or more anchoring elements 714, which may be similar to the anchoring elements 110 of the support structure 100, illustrated and described herein.

In various examples, the linking elements 706 are configured to couple the first and second sections 702 and 704 together, as well as deform to allow the second section 704 to be nested within the first section 702. For instance, the linking elements 706 may be configured to invert as shown in FIG. 7B as the second section 704 is drawn into the first section 702.

In some examples, the region defined between the first and second sections 702 and 704 (e.g., along the linking elements 706) may be covered or filled with a graft material (e.g., such as any of the graft materials discussed here) to provide a seal between the first and second section 702 and 704. Further examples of suitable configurations of telescoping first and second sections similar to those described above is shown in FIG. 10 and further discussion of which can be found in application Ser. No. 16/129,779 (claiming priority to Provisional Application Ser. No. 62/579,762), entitled “TELESCOPING PROSTHETIC VALVE AND DELIVERY SYSTEM,” filed by Applicant and referred to above.

It should be appreciated that the ability to nest the second section 704 within the first section 702 provides that the first section 702 can be expanded to engage the tissue without compromising the ability of the second section 704 to engage and interface with the manual pump 200. That is, the interface between the manual pump 200 and the support structure 700 can be maintained and closely controlled to provide an effective seal therebetween regardless of the degree to which the first section 702 is expanded to engage the surrounding tissue.

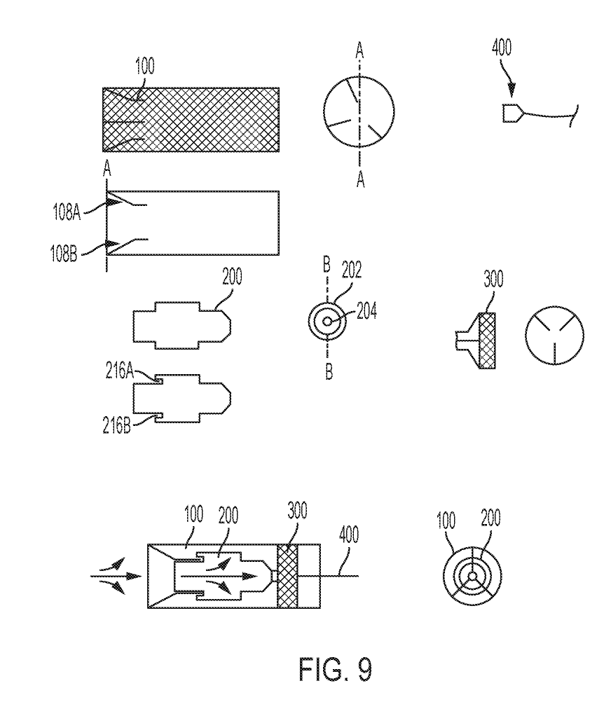

FIG. 9 shown a variety of additional configurations for the various components (e.g., the support structure 100, the pump 200, the retention element 300, and the driveline 400) of the systems disclosed herein. For instance, in some examples, the support structure 100 may include one or more support components (e.g., components 108a and 108b) that project radially inwardly and are configured to interface with and support the pump 200 within the support structure 100, as shown. In some examples, the pump 200 may include one or more features that are complementary of the support components 108a and 108b of the support structure 100, and that engage therewith to couple the pump 200 to the support structure 100, such that the pump 200 is suspended within an interior of the support structure 100 (e.g., within a lumen defined by an interior of the support structure 100). As shown, the pump 200 is coaxially aligned with the support structure 100, wherein an exterior of the pump 200 is offset from an interior of the support structure 100 such that an annular void is defined between the interior of the support structure 100 and the pump 200. In various examples, blood is operable to flow through such an annular void (e.g., in conjunction with, or as an alternative to blood flow through the pump 200).

FIG. 10 is a side view an example system 1000 in accordance with various aspects of the present disclosure. The support structure 100 is shown in a deployed configuration showing with the support structure 100 having a first frame subcomponent 1200 translated from a second frame subcomponent 1100, with an interstage 1302 therebetween in nested alignment. In certain instances, the frame subcomponent 1200 is nestable within the frame subcomponent 1100. The frame subcomponent 1100 and the frame subcomponent 1200 can be nested in-situ after the anchor frame subcomponent 1100 and the valve frame subcomponent 1200 are deployed at a treatment site in a patient's anatomy. The support structure may be delivered to a treatment region within a patient's anatomy with the frame subcomponent 1100 and the frame subcomponent 1200 longitudinally offset relative to one another and subsequently nested with one another at the treatment site.

In certain instances, the frame subcomponent 1100 and the frame subcomponent 1200 are operable to nest with one another by telescoping the frame subcomponent 1100 and the frame subcomponent 1200 relative to one another in-situ. Thus, in various examples, the frame subcomponent 1200 and the frame subcomponent 1100 are sized such that the frame subcomponent 1200 can be receive within the interior region of the frame subcomponent 1100. In addition to or alternative to telescoping relative to one another, the frame subcomponent 1100, the frame subcomponent 1200, and the film 1300 are each configured to be compressed or collapsed to a delivery profile and then reexpanded in-situ to provide for transcatheter delivery of the support structure 100. The support structure 100 may also be constricted with a sheath as described in detail above.

The pump 200, as described in detail above, may be removably coupled to the support structure 100 after the support structure 100 is deployed at the target location.

The interstage 1300 includes a conduit 1302 that couples to the frame subcomponents 1100, 1200. The conduit 1302 may comprise any suitable material known in the art. By way of example, the conduit 1302 may be a film, fabric, among others. Although the term “film” is use throughout this disclosure, it is understood that the term includes film, fabric, and other suitable materials.

Leaflets, not shown for clarity, may be coapt around the pump 200 to allow for blood flow and operate as a replacement for a patient's heart valve such as the aortic valve or mitral valve (a valve where there is positive flow). In other instances, the pump 200 may be configured to allow blood flow through the support structure 100 as the patient's heart beats with the rhythm of the heart. In these instances, the pump 200 increases flow as compared to if the pump 200 where not present along with the rhythm of the heart. In some examples, the valve or leaflets 1020 are coupled to an interior surface of one or both of the frame subcomponents 1100, 1200. In other examples, a film that comprises a leaflet is contained between the frame subcomponents 1100, 1200.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A medical device for improving or assisting cardiac function of a patient, the medical device comprising:
a support structure configured to extend across leaflets of a valve of the patient, the support structure having a delivery configuration and a deployed configuration; and
one or more locating features arranged within an interior of the support structure and configured to removably couple a pump to the support structure in the deployed configuration.

2. The medical device of claim 1, further including a pump is configured to drive blood flow through the support structure and supply blood flow to an aorta and couple to the one or more locating features of the support structure.

3. The medical device of claim 2, wherein the support structure is configured to removably couple the pump after the support structure is deployed from the delivery configuration to the deployed configuration and the pump includes one or more engagement elements configured to lock within the one or more locating features of the support structure.

4. The medical device of claim 3, wherein the pump and the support structure form a seal therebetween to stop blood flow between the pump housing and the support structure.

5. The medical device of claim 3, wherein the support structure is configured to suspend the pump within the support structure to allow blood flow about the pump.

6. The medical device of claim 2, wherein the support structure is configured to pin the leaflets to heart tissue in an open position to minimize interference with the pump.

7. The medical device of claim 2, further including a controller configured to power the pump and a drive line coupled to the pump and the controller and configured to deliver power to the pump.

8. The medical device of claim 7, wherein the drive line is configured to route through one of the left or right subclavian arteries.

9. The medical device of claim 1, wherein the support structure includes at least one of a stent and a graft configured to collapse to the delivery configuration and engage and interface with the leaflets of the valve upon expansion of the support structure to the deployed configuration.

10. A modular system for assisting cardiac function of a patient, the system comprising:
a support structure configured to deploy at a target treatment region within the patient, the support structure including one or more locating features arranged within an interior of the support structure;
a pump including one or more engagement elements configured to removably couple to the locating features of the support structure after deployment of the support structure at the target treatment region; and
a power source configured to power the pump to drive blood flow through the support structure.

11. The system of claim 10, wherein the power source includes a controller configured to power the pump and a drive line removably coupled to the pump and the controller configured to deliver the power to the pump.

12. The system of claim 11, wherein the drive line is configured to route through one of the left or right subclavian arteries and couple to the pump after deployment of the pump within the support structure.

13. The system of claim 10, wherein the power source includes an extracorporeal control system configured to control operation of the pump and to wirelessly power the pump.

14. The system of claim 13, wherein the extracorporeal control system includes a transcutaneous energy transmission system configured to wireless transmit energy to the pump.

15. The system of claim 14, wherein the pump includes an antenna configured to receive the transcutaneous energy transfer.

16. A method of improving or assisting cardiac function of a patient, the method comprising:
deploying a pump system across leaflets of a valve of the patient, the pump system including a support structure having one or more locating features arranged within an interior of the support structure and a pump including one more engagement features configured to removably couple to the one or more locating features of the support structure; and
operating the pump to drive blood flow through the support structure.

17. The method of claim 16, wherein the operating the pump includes driving blood across the pump and into an aorta.

18. The method of claim 17, wherein operating the pump includes drawing blood from a left ventricle and across the pump into the aorta.

19. A method of delivering a medical device for assisting cardiac function of a patient to a target location, the method comprising:
deploying a support structure at a target treatment region within the patient, the support structure including one or more locating features arranged within an interior of the support structure;
arranging a pump within the support structure, the pump including one or more engagement elements; and
coupling the pump to the support structure by engaging the locating features of the support structure and the engagement elements of the pump.

20. The method of claim 19, further comprising coupling a power source to the pump.

21. The method of claim 20, wherein the power source includes a controller configured to power the pump and coupling the power source to the pump includes coupling a drive line to the pump and the controller configured to deliver the power to the pump.

22. The method of claim 21, wherein coupling the drive line to the pump includes routing the driveline through one of the left or right subclavian arteries and couple to the pump after coupling the pump to the support structure.

23. The method of claim 20, wherein the coupling a power source to the pump includes wirelessly coupling an extracorporeal control system to the pump to control operation of the pump and to wirelessly power the pump

24. The method of claim 19, wherein deploying the pump system includes arranging the support system through one of a femoral access, a subclavian access, or transcaval access.
